SEQUENCE LISTING (1 sequence)

SEQ ID NO: 1 - DNA, 4963 bp, HOMO SAPIENS
agcgcggcgctgcagccatggcgggcggcgtgcggctgctctgggtgtcgctattggtgctgctggcgcagctagggccgcagcctggactgggccggctcggagagcgtctccgcgtgcgcttcaccccggtcgtgtgcggcctgcgctgcgtccatgggccgaccggctcccgctgtaccccgacctgcgcgccccgcaacgccaccagcgtggacagcggcgctcccggcggggcggccccggggggacccggcttccgcgccttcctgtgtcccttgatctgtcacaatggcggtgtgtgcgtgaagcctgaccgctgcctctgtcccccggacttcgctggcaagttctgccagttgcactcctcgggcgcccggcccccggccccggctataccaggcctcacccgctccgtgtacactatgccactggccaaccaccgcgacgacgagcacggcgtggcatctatggtgagcgtccacgtggagcacccgcaggaggcgtcggtggtggtgcaccaggtggagcgtgtgtctggcccttgggaggaggcggacgctgaggcggtggcgcgggcggaagcggcggcgcgggcggaggcggcagcgccctacacggtgttggcacagagcgcgccgcgggaggacggctactcagatgcctcgggcttcggttactgctttcgggagctgcgcggaggcgaatgcgcgtccccgctgcccgggctccggacgcaggaggtctgctgccgaggggccggcttggcctggggcgttcacgactgtcagctgtgctccgagcgcctggggaactccgaaagagtgagcgccccagatggaccttgtccaaccggctttgaaagagttaatgggtcctgcgaagatgtggatgagtgcgcgactggcgggcgctgccagcacggcgagtgtgcaaacacgcgcggcgggtacacgtgtgtgtgccccgacggctttctgctcgactcgtcccgcagcagctgcatctcccaacacgtgatctcagaggccaaagggccctgcttccgcgtgctccgcgacggcggctgttcgctgcccattctgcggaacatcactaaacagatctgctgctgcagccgcgtaggcaaggcctggggccggggctgccagctctgcccacccttcggctcagagggtttccgggagatctgcccggctggtcctggttaccactactcggcctccgacctccgctacaacaccagacccctgggccaggagccaccccgagtgtcactcagccagcctcgtaccctgccagccacctctcggccatctgcaggctttctgcccacccatcgcctggagccccggcctgaaccccggcccgatccccggcccggccctgagtttcccttgcccagcatccctgcctggactggtcctgagattcctgaatcaggtccttcctccggcatgtgtcagcgcaacccccaggtctgcggcccaggacgctgcatttcccggcccagcggctacacctgcgcttgcgactctggcttccggctcagcccccagggcacccgatgcattgatgtggacgaatgtcgccgcgtgcccccgccctgtgctcccgggcgctgcgagaactcaccaggcagcttccgctgcgtgtgcggcccgggcttccgagccggcccacgggctgcggaatgcctggatgtggacgagtgccaccgcgtgccgccgccgtgtgacctcgggcgctgcgagaacacgccaggcagcttcctgtgcgtgtgccccgccgggtaccaggctgcaccgcacggagccagctgccaggatgtggatgaatgcacccagagcccaggcctgtgtggccgaggggcctgcaagaacctgcctggctctttccgctgtgtttgcccggctggcttccggggctcggcgtgtgaagaggatgtggatgagtgtgcccaggagccgccgccctgtgggcccggccgctgtgacaacacggcaggctcctttcactgtgcctgccctgctggcttccgctcccgagggcccggggccccctgccaagatgtggatgagtgtgcccgaagccccccaccctgcacctacggccggtgtgagaacacagaaggcagcttccagtgtgtctgccccatgggcttccaacccaacgctgctggctccgagtgcgaggatgtggatgagtgtgagaaccacctcgcatgccctgggcaggagtgtgtgaactcgcccggctccttccagtgcagggcctgtccttctggccaccacctgcaccgtggcagatgcactgatgtggacgaatgcagttcgggtgcccctccctgtggtccccacggccactgcactaacaccgaaggctccttccgctgcagctgcgcgccaggctaccgggcgccgtcgggtcggcccgggccctgcgcagacgtgaacgagtgcctggagggcgatttctgcttccctcacggcgagtgcctcaacactgacggctcctttgcctgtacttgtgcccctggctaccgacccggaccccgcggagcctcttgcctcgacgttgacgagtgcagcgaggaggacctttgccagagcggcatctgtaccaacaccgacggctccttcgagtgcatctgtcctccgggacaccgcgctggcccggacctcgcctcctgcctcgacgtggacgaatgtcgcgagcgaggcccagccctgtgcgggtcgcagcgctgtgagaactctcccggctcctaccgctgtgtccgggactgcgatcctgggtaccacgcgggccccgagggcacctgtgacgatgtggatgagtgccaagaatatggtcccgagatttgtggagcccagcgttgtgagaacacccctggctcctaccgctgcacaccagcctgtgaccctggctatcagcccacgccagggggcggatgccaggatgtggacgaatgccggaaccggtccttctgcggtgcccacgccgtgtgccagaacctgcccggctccttccagtgcctctgtgaccagggttacgagggggcacgggatgggcgtcactgcgtggatgtgaacgagtgtgaaacactacagggtgtatgtggagctgccctgtgtgaaaatgtcgaaggctccttcctctgtgtctgccccaacagcccggaagagtttgaccccatgactggacgctgtgttcccccacgaacttctgctggcatgttcccaggctcgcagccccaggcacctgctagccccgttctgcccgccaggccacctccgccacccctgccccgccgacccagcacacctaggcagggccctgtggggagtgggcgccgggagtgctactttgacacagcggccccggatgcatgtgacaacatcctggctcggaatgtgacatggcaggagtgctgctgtactgtgggtgagggctggggcagcggctgccgcatccagcagtgcccgggcaccgagacagctgagtaccagtcattgtgccctcacggccggggctacctggcgcccagtggagacctgagcctccggagagacgtggacgaatgtcagctcttccgagaccaggtgtgcaagagtggcgtgtgtgtgaacacggccccgggctactcatgctattgcagcaacggctactactaccacacacagcggctggagtgcatcgacaatgacgagtgcgccgatgaggaaccggcctgtgagggcggccgctgtgtcaacactgtgggctcttatcactgtacctgcgagcccccactggtgctggatggctcgcagcgccgctgcgtctccaacgagagccagagcctcgatgacaatctgggagtgtgctggcaggaagtgggggctgacctcgtgtgcagccaccctcggctggaccgtcaggccacctacacagagtgctgctgcctgtatggagaggcctggggcatggactgcgccctctgccctgcgcaggactcagatgacttcgaggccctgtgcaatgtgctacgcccccccgcatatagccccccgcgaccaggtggctttggactcccctacgagtacggcccagacttaggtccaccttaccagggcctcccatatgggcctgagttgtacccaccacctgcgctaccctacgacccctacccaccgccacctgggcccttcgcccgccgggaggctccttatggggcaccccgcttcgacatgccagactttgaggacgatggtggcccctatggcgaatctgaggctcctgcgccacctggcccgggcacccgctggccctatcggtcccgggacacccgccgctccttcccagagcccgaggagcctcctgaaggtggaagctatgctggttccctggctgagccctacgaggagctggaggccgaggagtgcgggatcctggacggctgcaccaacggccgctgcgtgcgcgtccccgaaggcttcacctgccgttgcttcgacggctaccgcctggacatgacccgcatggcctgcgttgacatcaacgagtgtgatgaggccgaggctgcctccccgctgtgcgtcaacgcgcgttgcctcaacacggatggctccttccgctgcatctgccgcccaggatttgcacccacgcaccagccacaccactgtgcgcccgcacgaccccgggcctgagccctggcacccgatggccacccacccgcgcccgccactcggggcccctgccccgcatcctgcagcccgcttagtctgatgacgaggaagcccgccagaaagtccagaagaaggaacgacggacgcaaagcggcgccgcctaccatgcctcccccccccaccaccaccccccccaactgtggtcgtccccgcccggcccaccccgcccccatttctccccccttctttcaataaaaatttcaatcataaaaaaccacctataaaaaaaaaaa

(19) United States
(12) Patent Application Publication
(10) Pub. No.: US 2012/0196282 A1
(43) Pub. Date: Aug. 2, 2012

(54) METHOD OF REGENERATING ELASTIC FIBER AND SCREENING METHOD

(76) Inventors: Tomoyuki NAKAMURA, Moriguchi-shi (JP); Kyoko Takagi, Osaka-shi (JP); Kiyoshi Takayama, Moriguchi-shi (JP)

(21) Appl. No.: 13/015,871

(22) Filed: Jan. 28, 2011

(51) Int. Cl. C12Q 1/68; G01N 33/68; C12N 5/071

(52) U.S. Cl. 435/6.11; 435/375; 435/7.94

(57) ABSTRACT

A method of regenerating an elastic fiber according in the present invention is characterized in that the method comprises bringing an elastic fiber regenerating agent containing LTBP-4 into contact with a cell having the fiber regenerating ability. Preferably, the elastic fiber regenerating agent further contains DANCE and/or LTBP-4 expression potentiating factor. According to the present invention, the elastic fiber regenerating ability is further enhanced than so far.

### BACKGROUND OF THE INVENTION

1. Field of the Invention

The present invention relates to a method of regenerating an elastic fiber using an elastic fiber regenerating agent containing LTBP-4, and a method of screening an elastic fiber regenerating ability using LTBP-4 as an index.

2. Description of the Related Art

Elasticity (a nature of returning to the original state even when stretched) is seen in many tissues of a body, and is an essential nature for the function of tissues rich in stretchable tissues such as lung, artery and skin. What is responsible for elasticity in these tissues is an extracellular fiber called elastic fiber. It is thought that the elastic fiber is generated by sedimentation and crosslinking of an elastin protein at a periphery of a fiber called microfibril. The elastic fiber is deteriorated and torn by aging, or with a protease secreted from an inflammatory cell, which becomes a direct cause for sagging of a skin, and for lung emphysema, sclerosis of arterial medium and aortic aneurysm, and age-related macular degeneration etc., which are main diseases of an elder.

It is known that the elastic fiber is very slow in turnover and, even when the elastic fiber is deteriorated and torn, regeneration does not sufficiently occur. If regeneration of the elastic fiber becomes possible, it is expected that the generation can be applied to prevention and treatment of the skin sagging and aging-associated disease, but under the current circumstances, the molecular mechanism of formation of the elastic fiber has not been sufficiently studied yet.

The present inventors have been studying the formation mechanism and regeneration of the elastic fiber and, until now, have found out that a secreted protein called DANCE (developmental arteries and neural crest epidermal growth factor (EGF)-like; also referred to as another name “fibulin-5”) and/or fibulin-4 is essential for formation of the elastic fiber and, when a recombinant protein of them is added to a serum free medium of human skin fibroblast, formation and regeneration of the elastic fiber are promoted; and that when TGFβ (transforming growth factor β, see Schiemann et al., J. Biol Chem 277(30): 27367-77 (2002)) which enhances production of DANCE is added, formation and regeneration of the elastic fiber are promoted, and have reported them (Nakamura T et al., DANCE, a novel secreted ROD protein expressed in developing, atherosclerotic, and balloon-injured arteries, J. Biol. Chem. 274: 22476-83 (1999); Nakamura T et al., Fibulin-5/DANCE is essential for elastogenesis in vivo. Nature 415: 171-5 (2002); Hirai M, Ohbayashi T, Horiguchi M, Okawa K, Hagiwara A, Kita T, Chem K R, Nakamura T., Fibulin-5/DANCE has an elastogenic organizer activity that is abrogated by proteolytic cleavage in vivo. J Cell Biol 176: 1061-1071 (2007); WO 2005/093057 pamphlet; and WO 2006/082763 pamphlet).

On the other hand, Sterner-Kock A et al., Disruption of the gene encoding the latent transforming growth factor-beta binding protein 4 (LTBP-4) causes abnormal lung development, cardiomyopathy, and colorectal cancer. Genes Dev. 2002 Sep. 1; 16(17): 2264-73 and Dabovic B et al., Dual functions for LTBP in lung development: LTBP-4 independently modulates elastogenesis and TGF-beta activity. J Cell Physiol. 2009 April; 219(1): 14-22 report that abnormality of formation of the elastic fiber was seen in lung and intestinal tract, in a knockout mouse of LTBP-4 (latent TGFβ-binding protein-4). However, in the documents, how LTBP-4 is involved in regeneration of the elastic fiber was not studied at all.

As described above, DANCE is useful as an elastic fiber regenerating agent, but there was a problem that DANCE alone is not sufficient in the elastic fiber formation promoting activity, particularly, in the presence of serum. According to the result of study by the present inventors, in culturing of human skin fibroblast in the presence of serum, the elastic fiber was formed even without addition of DANCE and, even when DANCE was added thereto, no great change was seen. That is, in the presence of serum, formation of the elastic fiber was changed little, depending on the presence and the absence of addition of DANCE. Since tissues in a living body are perfused with blood, it is thought that they are under the condition similar to that of culturing in the presence of serum. Therefore, in view of utilization in a living body, provision of a regenerating agent which can exert the strong elastic fiber regenerating activity, not only under serum free, but also in the presence of serum, has been strongly desired.

The present invention has been made in view of the aforementioned circumstances, and an object thereof is to provide a method of regenerating using a novel elastic fiber regenerating agent, having the elastic fiber regenerating ability which has been further enhanced than previously. Further, another object of the present invention is to provide a substance constituting the elastic fiber generating agent, and a method of screening the elastic fiber regenerating ability.

### SUMMARY OF THE INVENTION

A method of regenerating an elastic fiber according to the present invention which solves the above-mentioned problems is characterized in that the method comprises bringing an elastic fiber regenerating agent containing LTBP-4 into contact with a cell having the fiber regenerating ability.

In the preferable embodiment, the elastic fiber regenerating agent further contains DANCE.

In the preferable embodiment, the elastic fiber regenerating agent further contains a LTBP-4 expression potentiating factor and/or a DANCE expression potentiating factor, represented by TGFβ etc.

In the preferable embodiment, the elastic fiber regenerating agent is contacted with the cell in the presence of serum.

Also, a method of screening a LTBP-4 expression potentiating factor according to the present invention which solves the above-mentioned problems is characterized in that the method comprises using an expression amount of LTBP-4 or a mRNA encoding LTBP-4 as an index.

Also, a method of screening an elastic fiber regenerating ability according to the present invention which solves the above-mentioned problems is characterized in that the method comprises using an expression amount of LTBP-4 or a mRNA encoding LTBP-4 as an index. In the screening method, it is preferable to use an expression amount of DANCE or a mRNA encoding DANCE is further used as an index.

The present invention includes an elastic fiber regenerating agent containing LTBP-4. Preferably, the elastic fiber regenerating agent further contains DANCE. The elastic fiber regenerating agent further may contain a LTBP-4 expression potentiating factor and/or a DANCE expression potentiating factor, represented by TGFβ etc.

The present invention includes a medicament containing the elastic fiber regenerating agent as an active ingredient. The medicament further may contain DANCE. Also, the medicament further may contain a LTBP-4 expression potentiating factor and/or a DANCE expression potentiating factor, represented by TGF etc.

When the LTBP-4-containing elastic fiber regenerating agent of the present invention is used, the regenerating ability is equal, or enhanced more as compared with the previous DANCE-containing regenerating agent. The elastic fiber regenerating activity according to the present invention is exerted regardless of the presence or the absence of serum and, under serum free, particularly, when a regenerating agent containing both of LTBP-4 and DANCE is used, regeneration of the elastic fiber is dramatically promoted as compared with the case where a regenerating agent of LTBP-4 alone or DANCE alone is used. In addition, little activity of the previous DANCE-containing regenerating agent in the presence of serum is recognized, while when the LTBP-4-containing elastic fiber regenerating agent of the present invention is used, formation of the elastic fiber in the presence of serum can be remarkably promoted and the effect was demonstrated even in vivo.

### BRIEF DESCRIPTION OF THE DRAWINGS

FIG. 1 is a photograph when purified recombinant human LTBP-4 (about 180 kD) and recombinant human DANCE (about 58 kD) are developed with SDS-PAGE and, thereafter, CBB-stained.

FIG. 2 is a photograph of an elastic fiber when human skin fibroblast is cultured in the presence of serum, which is fluorescently immunologically stained, and shows photographs of a control; of the case where LTBP-4 is knocked down with a siRNA; of the case where recombinant LTBP-4 is added thereto, respectively, in an order from an upper side.

FIG. 3 is a photograph of an elastic fiber when human skin fibroblast is cultured on a serum free medium, which is fluorescently immunologically stained, and shows photographs of the case where no protein is added; of the case where recombinant DANCE is added; of the case where recombinant LTBP-4 is added; and the case where these are added at the same time, respectively, in an order from an upper side.

FIG. 4 is a photograph showing the result when DANCE with a FLAG tag attached thereto, and LTBP-4 with a Myc tag attached thereto are mixed, and the mixture was immunoprecipitated with anti-FLAG antibody agarose.

FIG. 5 is a graph showing the amounts (ng) of desmosine and isodesmosine relative to each 1 μg of L-methionine for each of the test substance-treated group and the control substance-treated group in vivo experiment of LTBP-4 plus DANCE.

FIG. 6 is a graph showing the amounts (ng) of desmosine and isodesmosine relative to each 1 μg of L-valine for each of the test substance-treated group and the control substance-treated group in vivo experiment of LTBP-4 plus DANCE.

FIG. 7 is a graph showing the amounts (ng) of desmosine and isodesmosine relative to each 1 μg of L-tyrosine for each of the test substance-treated group and the control substance-treated group in vivo experiment of LTBP-4 plus DANCE.

FIG. 8 is a photograph showing the result of EVG staining in vivo experiment of LTBP-4 plus DANCE, and shows photographs on the left side for the test substance-treated group and on the right side for the control substance-treated group.

### DETAILED DESCRIPTION OF THE PREFERRED EMBODIMENTS

The present inventors have continued to study, in order to provide a new method of regenerating an elastic fiber, the ability to regenerate an elastic fiber of which is further enhanced, as compared with the previous DANCE-containing elastic fiber. As a result, they have found out that when an elastic fiber is regenerated using a LTBP-4-containing elastic fiber regenerating agent including LTBP-4, the superior activity is exerted regardless of the presence or the absence of serum, resulting in completion of the present invention.

Particularly, it has been found out that LTBP-4 binds with DANCE; knockdown of a LTBP-4 gene in culturing of human skin fibroblast makes it impossible to form the elastic fiber even in the presence of serum; when recombinant LTBP-4 is added to this culturing system, an extremely large amount of the elastic fiber is formed; and the elastic fiber forming activity due to LTBP-4 is synergistically promoted by joint use of DANCE. In the fibroblast culturing system of the presence of serum, the activity of promoting formation of the elastic fiber is confirmed first in LTBP-4, and this is the prominent finding which has not been seen in the previous DANCE-containing elastic fiber regenerating agent. In addition, dramatic promotion of formation of the elastic fiber not only in the presence of serum but also under serum free, by joint use of LTBP-4 and DANCE, is the finding which far exceeds the previous prediction.

In the presence specification, “regenerating” in the “method of regenerating an elastic fiber” means bringing the elastic fiber lost by deterioration and tearing of the elastic fiber into the state where the fiber can be regenerated (neogenerated), and regeneration also includes formation. Therefore, formation promotion of insufficient formation of the elastic fiber is also included.

(Method of Regenerating Elastic Fiber)

A method of regenerating the elastic fiber in the present invention has the characteristic in that the elastic fiber is regenerated by bringing the elastic fiber regenerating agent containing LTBP-4 into contact with a cell having the ability to regenerate a fiber. The contacting method includes both of in vitro and in vivo methods. The regenerating method of the present invention is substantially the same as the regenerating method described in the aforementioned patent publication 2 (WO 20061082763 pamphlet) except that the elastic fiber regenerating agent containing LTBP-4 is used Details are as follows.

First of all, it explains the elastic fiber regenerating agent that characterizes the present invention most.

The elastic fiber regenerating agent includes at least LTBP-4 as an active ingredient and, preferably, further includes DANCE and/or a known LTBP-4 expression potentiating factor or DANCE expression potentiating factor. The DANCE-containing elastic fiber regenerating agent described in the aforementioned patent publication 2 (WO 2006/082763 pamphlet) (previous example) is different from the LTBP-4-containing elastic fiber regenerating agent used in the present invention in that it does not include LTBP-4. The regenerating agent used in the present invention can be applied to, for example, medicaments, reagents for cultured cells, or artificial tissues etc., as detailed below.

LTBP-4 used in the present invention refers to a polypeptide derived from a base sequence described in SEQ ID No.: 1 (GenBank accession number AF051344) or an orthologue thereof, or a variant thereof (SNP, including haplotype). The orthologue of LTBP-4 is not particularly limited, but can be derived from, for example, an arbitrary animal, preferably a mammal. Examples of the mammal include cow, sheep, pig, goat, monkey, rabbit, rat, hamster, guinea pig and mouse etc. LTBP-4 is a secreted protein, and a signal sequence (e.g., MAGGVRLLWVSLLVLLAQLGPQPGLG) can be removed by processing. In the method of the present invention, as LTBP-4, any of LTBP-4 whose signal sequence has been removed, and LTBP-4 whose signal sequence has not been removed can be used, and LTBP-4 whose signal sequence has been removed is preferable. In addition, the LTBP-4 variant is not particularly limited as far as it enables regeneration of the elastic fiber, and examples include polypeptides which hybridize with a DNA having a sequence complementary with a base sequence shown in SEQ ID No.: 1 under the stringent condition, and are derived from a DNA encoding LTBP-4.

The regenerating agent used in the present invention may further include DANCE. As verified in Example described later, since when the regenerating agent containing both of LTBP-4 and DANCE is used under serum free, regeneration of the elastic fiber was dramatically promoted as compared with the case where the regenerating agent of LTBP-4 alone or DANCE alone is used, the regenerating agent further including DANCE is extremely useful for regeneration of the elastic fiber. DANCE used in the present invention is detailed in the aforementioned patent publication 1 (WO 2005/093057 pamphlet), and the aforementioned patent publication 2 (WO 2006/082763 pamphlet), and an amino acid sequence described in SEQ ID No.: 2 or 4 of WO 2005/093057 pamphlet, or an equivalent thereof etc. is used. Alternatively, as described in WO 2006/082763 pamphlet, an amino acid sequence whose signal sequence (corresponding to a sequence of first to 23″ amino acids in an amino acid sequence described in SEQ ID No.: 1 of the aforementioned patent publication 2 (WO 2006/082763 pamphlet) has been removed by processing may be used. Alternatively, it is confirmed that DANCE is cut with a DANCE-specific protease as described in the aforementioned patent publication 1 (WO 2005/093057 pamphlet), and the DANCE variant described in the aforementioned patent publication 2 (WO 2006/082763 pamphlet) may also be used.

The regenerating agent used in the present invention may further contain a LTBP-4 expression potentiating factor (LTBP-4 induction factor) and/or a DANCE expression potentiating factor (DANCE induction factor). In the present invention, the known LTBP-4 expression potentiating factor and/or DANCE expression potentiating factor which are known to potentiate expression of LTBP-4 can be used, and examples include a LTBP-4 expression potentiating factor such as TGFβ; a DANCE expression potentiating factor such as a caesalpinia crista extract, a psophocarpus tetragonolobus extract, phytic acid, and a physiologically acceptable salt thereof, as well as L-hydroxyproline and a physiologically acceptable salt thereof; a LTBP-4 expression vector or a DANCE expression vector, or a LTBP-4 induction factor expression vector or a DANCE induction factor expression vector (details of which will be described later) etc. As the TGFβ, TGFβ-1 etc. is representatively exemplified.

The regenerating agent used in the present invention may further include, in addition to the aforementioned active ingredient, a pharmaceutically acceptable salt thereof. Specifically, when the active ingredient has an acidic group such as a carboxyl group, examples of a salt of the active ingredient include salts of alkali metals and alkaline earth metals such as lithium, sodium, potassium, magnesium and calcium; salts of ammonia, or amines such as methylamine, dimethylamine, trimethylamine, dicyclohexylamine, tris(hydroxymethyl)aminomethane, N,N-bis(hydroxyethyl)piperazine, 2-amino-2-methyl-1-propanol, ethanolamine, N-methylglucamine and L-glucamine; or salts with basic amino acids such as lysine, δ-hydroxylysine and arginine. In addition, when the active ingredient has a basic group such as an amino group, examples of a salt of the active ingredient include salts of mineral acids such as hydrochloric acid, hydrobromic acid, sulfuric acid, nitric acid and phosphoric acid; salts with organic acids such as methanesulfonic acid, beuzenesulfonic acid, paratoluenesulfonic acid, acetic acid, propionic acid, tartaric acid, fumaric acid, maleic acid, malic acid, oxalic acid, succinic acid, citric acid, benzoic acid, mandelic acid, cinnamic acid, lactic acid, glycolic acid, glucuronic acid, ascorbic acid, nicotinic acid and salicylic acid; or salts with acidic amino acids such as aspartic acid and glutamic acid.

Alternatively, the regenerating agent used in the present invention may include a solvate or a hydrate of the pharmaceutically acceptable salt.

The regenerating agent used in the present invention may be constructed of only the active ingredient, but for example, when used in medicaments, it may include pharmaceutically acceptable arbitrary carries (additives). The “pharmaceutically acceptable carrier” is not particularly limited, but any of inorganic substances or organic substances or any carries which are solid or liquid can be used as far as they are conventional. Herein, examples of the pharmaceutically acceptable carrier are not limited to, but include excipients such as sucrose, starch, mannit, sorbit, lactose, glucose, cellulose, talc, calcium phosphate and calcium carbonate; binders such as cellulose, methylcellulose, hydroxypropylcellulose, polypropylpyrrolidone, gelatin, gum arabic, polyethylene glycol, sucrose and starch; disintegrating agents such as starch, carboxymethylcellullose, hydroxypropylstarch, sodium-glycol-starch, sodium bicarbonate, calcium phosphate and calcium citrate; lubricants such as magnesium stearate, aerosil, talc and sodium laurylsulfate; aromatic agents such as citric acid, menthol, a glycyllysine ammonium salt, glycine and an orange powder; preservatives such as sodium benzoate, sodium hydrogen sulfite, methylparaben and propylparaben; stabilizers such as citric acid, sodium citrate and acetic acid; suspending agents such as methylcellulose, polyvinylpyrrolidone and aluminum stearate; dispersants such as a surfactant; diluents such as water, physiological saline and an orange juice; base waxes such as cacao butter, polyethylene glycol and kerosene. These can be blended at a ratio of approximately 1 part by mass to 90 parts by mass based on 100 parts by mass of the active ingredient.

An administration form used in the regenerating agent in connection with the present invention is not limited, but any of oral administration and parenteral administration can be arbitrarily selected depending on therapeutic purpose etc. An administration form of oral administration and parenteral administration is not particularly limited, as far as it is pharmaceutically acceptable,

Among them, examples of a dosage form suitable for oral administration include liquid preparations in which an effective amount of a ligand is dissolved in a diluent such as water, physiological saline and an orange juice; capsules including an effective amount of a ligand as a solid or a granule (hard capsules, soft capsules); sachets or tablets; suspension preparations in which an effective amount of a ligand is suspended in an appropriate dispersing medium; and emulsions in which a solution with an effective amount of a ligand dissolved therein is dispersed and emulsified in an appropriate dispersing medium. Besides, examples include granules, fine granules, powders and syrups. These are formulated into preparations according to the conventional method. In addition, tablets and granules etc. may be coated by the well-known methods.

A method for producing solid preparations for oral administration is not particularly limited, but the conventional method may be applied. Specifically, examples include a method of mixing an active ingredient and an excipient ingredient (e.g., lactose, starch, crystalline cellulose, calcium lactate and silicic acid anhydride) into powders, or further, if necessary, adding a binder such as sucrose, hydroxylpropylcellulose and polyvinylpyrrolidone; a disintegrating agent such as carboxymethylcellulose and carboxymethylcellulose calcium, followed by wet or dry granulation into granules. In addition, for producing tablets, these powders or granules may be compressed as they are, or by adding a lubricant such as magnesium stearate and talc. These powders, granules, or tablets may be covered with an enteric coated agent base such as hydroxypropylmethylcellulose phthalate and a methacrylic acid-methyl methacrylate polymer into enteric coated preparations, or may be covered with ethylcellulose, carnauba wax or a hardened oil into depot preparations. In addition, for producing capsules, the powders or granules can be filled into hard capsules, or an active ingredient as it is, or after it is dissolved in glycerin, polyethylene glycol, a sesame oil or an olive oil, can be covered with a gelatin film into soft capsules.

Meanwhile, examples of a preparation suitable for parenteral administration (e.g., intravenous injection, subcutaneous injection, intramuscular injection, local injection and intraperitoneal administration) include aqueous and non-aqueous isotonic sterile injection liquid preparations, aqueous and non-aqueous sterile suspension preparations, drops, suppositories, ointments, cream preparations, gel preparations, patch preparations, inhalants, drops, transdermal absorbents, transmucosal absorbents and nose drops. These preparations may include conventional additives. For example, injection liquid preparations may include antioxidants, buffers, bacteriostatic agents, tonicity agents and preservatives, and suspension liquid preparations may include suspending agents, solubilizers, thickeners, stabilizers and antiseptics.

These are formulated into preparations according to the conventional method. For example, injection liquid preparations are prepared by dissolving the active ingredient in water and, if necessary, the active ingredient may be dissolved in a pH adjusting agent such as hydrochloric acid, sodium hydroxide, lactose, lactic acid, sodium, sodium monohydrogen phosphate and sodium dihydrogen phosphate, or a tonicity agent such as sodium chloride and glucose. In addition, such as injection preparations or drops are prepared as a powdery dosage form such as a lyophilized form and may be prepared by dissolving it in an appropriate aqueous medium such as a physiological saline upon use. Alternatively, a sustained release-preparation covered with a polymer etc. can be also directly administered into a brain. In addition, suspension preparations can be sealed into a container by a unit dose or a plurality of doses, like ampoules or vials. In addition, an active ingredient and a pharmaceutically acceptable carrier are lyophilized, and can be also stored in the state where it may be dissolved or suspended in an appropriate sterile vehicle immediately before use.

More particularly, for example, in order to produce injection preparations, an active ingredient, if necessary, together with the tonicity agent is dissolved in distilled water for injection, sterilely filtered and may be filled into ampoules, or further mannitol, dextrin, cyclodextrin or gelatin etc. may be added thereto, and this may be vacuum-lyophilized into injection preparations which are soluble upon use. Alternatively, lecithin, Polysolvate 80, or polyoxyethylene hardened castor oil etc. may be added to an active ingredient to emulsify the ingredient in water, into emulsions for injection preparations.

In addition, in order to produce rectal administration preparations, an active ingredient together with a base for suppositories such as cacao butter, tri-, di- and mono-glyceride of fatty acid, and polyethylene glycol is wetted to dissolve the active ingredient, and the solution is cast into a mold, and cooled; or an active ingredient is dissolved in polyethylene glycol or a soybean oil etc. and, thereafter, may be covered with a gelatin film.

In addition, in order to produce external preparations for skin, an active ingredient is added to white vaseline, beeswax, liquid paraffin or polyethylene glycol etc. and, if necessary, this is wetted to knead it into ointments; or an active ingredient is kneaded with an adhering agent such as rosin and an acrylic acid alkyl ester polymer and, thereafter, this may be spread on a non-woven fabric such as polyalkyl into tape preparations.

In addition, the regenerating agent used in the present invention can also be used as a sustained-release preparation for a delivery system, which is sealed into an implant tablet and a microcapsule. This sustained-release preparation can be prepared using the pharmaceutically acceptable known carrier, which can prevent rapid removal from the interior of a body. Specifically, a biodegradable or biocompatible polymer such as ethylene vinyl acetate, polyacid anhydride, polyglycolic acid, collagen, polyorthoester, and polylactic acid can be used. These polymers are known, and can be easily prepared by a person skilled in the art based on the conventional method. Alternatively, as the pharmaceutically acceptable other carrier, a suspension such as a liposome can also be used. The liposome is not particularly limited, but examples include such as lipid compositions containing phosphatidylcholine, cholesterol and PEG-derived phosphatidylethanol (PEG-PE). This is obtained by preparing it by passing through a filter having an appropriate pore size so that the size suitable for use is obtained, and purifying this by a reverse phase evaporation method.

A dose of the regenerating agent used in the present invention may be appropriately selected, in view of a kind and activity of an active ingredient, severity of a disease, an animal species which is an administration subject, and a weight, an age and drug acceptability of an administration subject etc., and it is usually preferable that a content of an active ingredient per adult a day is about 0.05 to about 100 mg/kg.

In addition, when the regenerating agent used in the present invention is used in medical utility, it may be used in a form of a kit. Specifically, examples include an aspect in which different kinds of constituent ingredients constituting a pharmaceutical composition are wrapped in separate containers or packs in advance, and they are mixed immediately before use. Thereby, storage for a long term becomes possible without losing the function of various constituent ingredients. Examples of a usable container include containers which can effectively maintain the activities of various constituent ingredients for a long term, and do not adsorb ingredients, and do not undergo degeneration, depending on a material of containers. Examples of the container include a sealed glass ampoule, and this includes a buffer wrapped under a neutral non-reactive gas such as a nitrogen gas. This glass ampoule is constructed of a glass, an organic polymer such as polycarbonate and polystyrene, a ceramic, a metal or other appropriate material which is usually used for retaining constituent ingredients. Alternatively, examples of other appropriate container include a simple bottle made of a similar substance such as an ampoule, and a wrapping material in which the interior is backed with a foil of aluminum or an alloy. A form of the container is not particularly limited, but examples include test tubes, vials, flasks, bottles, syringes, or their similar forms. The container may have an aseptic access port, and examples include such as a bottle having a stopper which can be penetrated with a subcutaneous injection needle.

The kit may include a manual (package insert) for using the medicament. A form of the manual is not particularly limited, but for example, may be a form which is printed on paper or other material, or may be electrically or electromagnetically readable medium such as a FD, a CD-ROM, a DVD-ROM, a Zip disc, a videotape and an audiotape. It is not necessarily required that all detailed explanation is described on the manual, but for example may be posted on a website which is designated by a producer or a distributor of a kit, or is notified by an electric mail etc.

The regenerating agent used in the present invention is useful for preventing, treating or improving the state or a disease in which regeneration of the elastic fiber is desired, for example, lung emphysema, blood vessel damage, dermatolysis, wound, elastic fiber deterioration (e.g. deterioration which is caused by aging or ultraviolet ray), skin roughness, arteriosclerosis, aortic aneurysm, age-related macular degeneration, perineal hernia, anal hernia, basilar artery aneurysm, digestive tract movement disorder or bedsore etc. In addition, the regenerating agent used in the present invention is useful for beauty purpose such as prevention and improvement of skin sagging or wrinkle. In addition, the regenerating agent of the present invention is also useful such as a reagent for culturing a cell having the ability to regenerate the elastic fiber.

When the regenerating agent used in the present invention is used, an artificial elastic fiber including LTBP-4, preferably both of LTBP-4 and DANCE can be provided. Herein, the “artificial elastic fiber” means an elastic fiber which has been regenerated in vitro. The artificial elastic fiber may not include LTBP-4 used for culturing, and/or an ingredient derived from an animal which is heterogeneous to an animal from which a cell having the ability to regenerate an elastic fiber used for culturing is derived. That is, the artificial elastic fiber can be one consisting of only ingredients derived from a single animal species for biological ingredients derived from animals. According to the method of the present invention, it has first become possible to provide such an artificial elastic fiber. Therefore, the artificial elastic fiber can be suitably used in homogeneous transplantation such as allogeneic transplantation. In addition, by using a cell derived from an individual for which transplantation is desired, as a cell having the ability to regenerate an elastic fiber, and using a medium in which a heterogeneous animal-derived ingredient is not mixed in at all, an elastic fiber which is suitably used in syngeneic transplantation is obtained.

The artificial elastic fiber can also include an elastic fiber constituent ingredient other than LTBP-4 and DANCE (e.g., elastin, lysyloxidase (LOX), lysyloxidase-like 1 to 4 (LOXL 1 to 4), LTBP2, fibrillin 1 to 3, EMILIN1 and 2, MAGP 1 and 2), as well as other ingredient (e.g., betaig-h3, 67 kD elastin-binding protein).

In addition, when the regenerating agent used in the present invention is used, an artificial tissue including an elastic fiber can be provided. Examples of the artificial tissue including an elastic fiber include an artificial skin, an artificial blood vessel (e.g., artificial artery), artificial lung, artificial uterine, and artificial intestinal tract, and the artificial skin and the artificial blood vessel are preferable. These artificial tissues can be made by the known method (e.g., see Long, J. L. et al. (2003), Matrix biology 22 (4): 339-50, Muraguchi, T. et al., (1994), Plastic and Reconstructive Surgery 93 (3): 537-44).

It explained the elastic fiber regenerating agent used in the present invention above.

Next, it explains a method of contacting the elastic fiber regenerating agent with a cell having the ability to regenerate an elastic fiber.

The “cell having the ability to regenerate an elastic fiber” refers to a cell which secretes elastic fiber constituent ingredients, and enables regeneration (neogenesis, formation) of the elastic fiber by being cultured in the presence of LTBP-4. The “cell having the ability to regenerate an elastic fiber” is a cell derived from an arbitrary animal, preferably derived from a mammal. Examples of the mammal include the same mammal as the mammal from which an orthologue of LTBP-4 is derived.

The “cell having the ability to regenerate an elastic fiber” may be either of a non-transgenic cell or a transgenic cell. Among them, examples of the “non-transgenic cell” include cells derived from tissues in which the elastic fiber is present, for example, skin, blood vessel (e.g., artery), lung, uterine, heart, kidney, pancreas, testis, ovary, small intestine, colon, or cartilage tissue. The “non-transgenic cell” can be also a cell such as fibroblast, a smooth muscle cell, an epithelial cell, and an endothelial cell. Preferably, a skin fibroblast or blood vessel smooth muscle cell is used. The “non-transgenic cell” may also be a primary cultured cell, a cell strain induced from a primary cultured cell, or a cell obtained by culturing an undifferentiated cell such as a stem cell etc. Such cells can be prepared by the known method. For example, Current Protocols in Cell Biology, John Wiley & Sons, Inc. (2001); Separation and Culturing of Functional Cell, Maruzen Shoten Co., Ltd. (1987) etc. can be referenced.

In addition, examples of the “transgenic cell” include transgenic cells in which one or two or more genes are introduced so that expression thereof is possible. When a cell being an introduction subject is a cell having no ability, or not having sufficient ability to regenerate an elastic fiber, a cell which has acquired the ability to regenerate an elastic fiber, or a cell having the improved ability to regenerate an elastic fiber can be made by introducing a vector which can express elastic fiber constituent ingredients and/or other factor which promotes formation of an elastic fiber into the cell. Of course, a vector which can express the “elastic fiber constituent ingredient” and/or “other factor” which promotes formation of an elastic fiber may be introduced to more improve the ability to regenerate an elastic fiber. The “elastic fiber constituent ingredient” and “other factor” are not particularly limited, but can be appropriately determined by a person skilled in the art depending on a kind of a cell. Specifically, examples of the “elastic fiber constituent ingredient” include elastin, lysyloxidase (LOX), lysyloxidase-like 1 to 4 (LOXL1 to 4), LTBP2 and 4, fibrillin) to 3, EMILIN1 and 2, MAGP1 and 2 etc., and examples of “other factor” which promotes formation of an elastic fiber include versican V3, and hyaluronidase etc. In addition, as a vector which can express the “other factor”, and a method of introducing the vector into a cell, the same vectors and method as those of the LTBP-4 expression vector and DANCE induction factor expression vector described later can be used.

The regenerating method of the present invention, when a method of contacting with a cell is in vitro, includes a step of performing culturing while the elastic fiber regenerating agent of the present invention is brought into contact with the “cell having the ability to regenerate an elastic fiber” (hereinafter, simply abbreviated as “cell” in some cases). A density of the cell which is seeded in a culturing medium is not particularly limited, but the cell may be seeded to subconfluent or confluent.

The culturing method is not particularly limited, as far as it is a method which can culture the cell in the presence of LTBP-4, and examples include (1) culturing in a medium with LTBP-4 added thereto, (2) culturing in a medium with a LTPB-4 induction factor added thereto, (3) culturing of a cell with a LTBP-4 expression vector and/or a LTBP-4 induction factor expression vector introduced therein, and (4) coculturing of a LTBP-4 expression cell and/or a LTBP-4 induction factor expression cell, and the “cell having the ability to regenerate an elastic fiber”.

In the case of (1), LTBP-4 to be added to a medium may be either of a natural protein or a recombinant protein. LTBP-4 can be prepared and purified by the known method.

Examples of a representative method of preparing LTBP-4 include such as (a) a method of recovering LTBP-4 from a living body sample (e.g., blood) containing LTBP-4, (b) a method of making a transformant by introducing a LTBP-4 expression vector into a host cell (e.g., Escherichia bacteria, Bacillus bacteria, yeast, insect cells, insects, and animal cells), and recovering LTBP-4 produced by the transformant, and (c) a method of synthesizing LTBP-4 by a cell-free system using rabbit reticulocyte lysate, wheat germ lysate, or Escherichia coli lysate etc.

In addition, examples of a representative method of purifying LTBP-4 include a method of utilizing solubility such as salting out and a solvent precipitation method; a method utilizing mainly a difference in a molecular weight, such as a dialysis method, an ultrafiltration method, a gel filtration method, and a SDS-polyacrylamide gel electrophoretic method; a method of utilizing a difference in a charge, such as ion exchange chromatography; a method of utilizing specific affinity, such as affinity chromatography, and use of an anti-LTBP-4 antibody; a method of utilizing a difference in hydrophobicity, such as reverse phase high performance liquid chromatography; a method of utilizing a difference in an isoelectric point such as isoelectric focusing electrophoresis; and a method of combination of them, etc.

An amount of LTBP-4 to be added to a medium is not particularly limited, as far as it enables regeneration of an elastic fiber, but for example, it is recommended to add LTBP-4 so that a final concentration in a culturing medium becomes preferably 0.1 to 100 μg/mL, more preferably 0.5 to 50 μg/mL, and further preferably 1 to 20 μg/mL.

In the case of (2), the LTBP-4 induction factor to be added to a medium is not limited, as far as it can induce expression of LTBP-4, but examples include TGFβ (e.g., TGFβ1). When the LTBP-4 induction factor is a protein, a natural protein or a recombinant protein (which can be prepared like LTBP-4) can be used. An amount of the LTBP-4 induction factor to be added to a medium is not particularly limited, as far as it induces LTBP-4 at an amount to such an extent that it enables regeneration of an elastic fiber, but for example, when TGFβ is used as the LTBP-4 induction factor, it is recommended to add TGFβ so that a final concentration in a culturing medium becomes preferably 0.5 to 1000 ng/mL, more preferably 1 to 100 ng/mL, and further preferably 5 to 40 ng/mL.

In the case of (3), the expression vector to be introduced into a cell may be a vector which can express LTBP-4 or the LTBP-4 induction factor. These expression vectors must be functionally linked to a promoter in which polynucleotide coding an objective protein can exert the promoter activity in the objective cell. The promoter used is not particularly limited, as far as it can function in an objective cell, but examples include virus promoters such as SV40-derived early promoter, cytomegalovirus LTR, Rous sarcoma virus LTR, MoMuLV-derived LTR, and adenovirus-derived early promoter; and constitutive protein gene promoters of a mammal, such as β-actin gene promoter, PGK gene promoter, and transferrin gene promoter, etc. The promoter may also be a promoter specific for cells having the ability to regenerate an elastic fiber, such as fibroblast-specific promoter (e.g., collagen promoter), and smooth muscle cell-specific promoter (e.g., SM22 promoter, α-smooth muscle actin promoter), etc.

The expression vector preferably contains a transcription termination signal, that is, a terminator region downstream of a polynucleotide encoding an objective protein. The expression vector can also further contain a selective marker gene for selecting a transformed cell (a gene imparting resistance to a drug such as tetracycline, ampicillin, kanamycin, hygromycin, and phosphinothricin, a gene complementing auxotrophic mutation etc.). A vector of a fundamental skeleton used as the expression vector is not particularly limited, but examples include plasmid vectors, as well as virus vectors such as retrovirus, adenovirus, adeno-associated virus, and Sendai virus vectors, etc.

A method of introducing the expression vector into the cell is not particularly limited, but can be performed by the known method such as an electroporation method, a calcium phosphate precipitation method, a microinjection method, and a method using a lipid such as a liposome and a cationic lipid etc. In addition, a part or all of the expression vector may be incorporated or may not be incorporated into a genome of a cell having the ability to form an elastic fiber. Incorporation of the expression vector into an intracellular genome is not particularly limited, but the known method such as a method of using retrovirus, and a method of using a targeting vector which enables homologous recombination can be used.

In the case of (4), a cell which coexists with the “cell having the ability to regenerate an elastic fiber” in a medium is not particularly limited, as far as it is an expression cell which can express LTBP-4 or the LTBP-4 induction factor. Examples of this expression cell include cells obtained by introducing an expression vector of LTBP-4 or the LTBP-4 induction factor into a host cell, and natural cells which express LTBP-4 or the LTBP-4 induction factor. As the natural cell expressing LTBP-4, cells derived from LTBP-4 expressing tissues (e.g., heart, kidney, pancreas, testis, ovary, small intestine, colon, cartilage, artery, lung, uterine, and skin) can be used. As the natural cell expressing the LTBF-4 induction factor, for example, in the case where TGFβ is intended as the LTBP-4 induction factor, cells derived from tissues such as leukocyte, bone, placenta, spleen, small intestine, colon, liver, kidney, heart, brain, bone marrow, and cartilage etc. can be used. The cell expressing LTBP-4 or the LTBP-4 induction factor may also be such as a primary cultured cell, a cell strain induced from a primary cultured cell, a cell obtained by culturing an undifferentiated cell such as a stem cell (e.g., differentiated cell), a commercially available cell strain, or a cell strain available from a cell bank. The cell expressing LTBP-4 or the LTBP-4 induction factor may also be a cell derived from an animal, which is homogenous to an animal of a cell having the ability to regenerate an elastic fiber.

Examples of the coculturing method include a method of culturing the “cell having the ability to regenerate an elastic fiber” and the cell expressing LTBP-4 or the LTBP-4 induction factor while they are physically in contact with each other. Alternatively, these cells may be cultured in the state where they are present in the same culturing system, but the cells themselves cannot be physically in contact with each other by isolation with a diaphragm through which traverse of substances is possible, and such a method is also included. Specifically, for example, a method of culturing by isolating both cells using a filter used in a usual cell culturing (e.g., culture insert).

A medium used in the regenerating method of the present invention is not particularly limited, but a MEM medium, a DMEM medium, and a RPMI1640 medium etc. can be used Ingredients which are usually added to a medium, such as amino acids, vitamins, lipids, antibiotics and buffers may be further added to the medium. A preferable pH of the medium is for example about 6 to 8, and a more preferable pH is about 6.5 to 7.5. A preferable culturing temperature is for example about 30 to 40° C., and a more preferable culturing temperature is about 37° C. A preferable culturing term is also not particularly limited, but may be appropriately adjusted so that a sufficient amount of the elastic fiber is obtained, depending on the culturing condition such as a medium.

The culturing may be performed in any of a serum free medium and a serum medium (containing, for example, 5% or less, 3% or less or 1% or less serum). For example, in view of prevention of mixing of unidentified ingredients, and alleviation of an infection risk etc., the culturing is preferably performed in a serum free medium. In addition, from a viewpoint of removal of a heterogeneous animal-derived ingredient to inhibit an allergic reaction, it is preferable that LTBP-4 used in culturing, a cell having the ability to regenerate an elastic fiber, and an optionally added other ingredient are unified by those derived from a homogenous animal.

A medium used in the culturing, for effectively forming an elastic fiber, can contain elastic fiber constituent ingredients other than LTBP-4 and DANCE (e.g., elastin, lysyloxidase (LOX), lysyloxidase-like 1 to 4 (LOXL1 to 4), LTBP2, fibrillin 1 to 3, EMILIN1 and 2, MAGP1 and 2), and/or other factors promoting formation of an elastic fiber (e.g., versican V3, hyaluronidase), as well as/or other ingredients (e.g., betaig-h3, 67 kD elastin-binding protein).

Culturing may be performed by two-dimensional culturing or three-dimensional culturing. Two-dimensional culturing or three-dimensional can be performed by the known method. For example, for techniques regarding three-dimensional culturing, Long, J. L. et al. (2003), Matrix biology 22(4): 339-50, Muraguchi, T. et al., (1994) Plastic and Reconstructive Surgery 93 (3): 537-44 can be referenced.

Whether the elastic fiber is regenerated or not can be confirmed by the known method. For example, it can be confirmed by collecting a part of the culture, and immunostaining it using an antibody to the elastic fiber constituent ingredient such as an anti-LTBP-4 antibody, and an anti-elastin antibody etc.

(Method of Screening LTBP-4 Expression Potentiating Factor or Elastic Fiber Regenerating Ability)

The present invention also includes a method of screening a LTBP-4 expression potentiating factor or elastic fiber regenerating ability, by analyzing the expression situation of LTBP-4 or a mRNA encoding LTBP-4 using LTBP-4 as an index. That is, by measuring the presence or the absence of expression of, or an expression amount of LTBP-4 or a mRNA encoding LTBP-4, by the known method, the presence or the absence of regeneration of the elastic fiber, and the ability to regenerate an elastic fiber can be indirectly assessed.

The screening method of the present invention is suitably used, for example, for assessing the LTPB-4 expression potentiating ability and the elastic fiber regenerating ability of a test substance. Specifically, when a test substance and a cell are brought into contact with each other and, if necessary, the cell is cultured, thereafter, an expression amount of LTBP-4 or a mRNA encoding LTBP-4 of a test group which has been brought into contact with the test substance, and an expression amount of LTBP-4 or a mRNA encoding LTBP-4 of a control group (control) which has not been brought into contact with the test substance are compared and studied, and expression of LTBP-4 or a mRNA encoding LTBP-4 is significantly recognized, or an expression amount is increased as compared with the control group, it can be assessed that the test substance has the LTBP-4 expression potentiating ability, or the elastic fiber regenerating ability.

Alternatively, the screening method may further include a step of measuring the presence or the absence of expression of, or an expression amount of DANCE or a in RNA encoding DANCE by the known method. This is because the elastic fiber regenerating agent used in the present invention above includes preferably both of LTBP-4 and DANCE. By using both of LTBP-4 and DANCE as an index like this, screening of a substance having the both expression potentiating abilities, or screening of the elastic fiber regenerating agent including the LTBP-4 expression potentiating factor and the DANCE expression potentiating factor can be effectively performed.

A method of analyzing the expression situation of LTBP-4 or a mRNA encoding LTBP-4, or DANCE or a mRNA encoding DANCE is not particularly limited, but the conventional method can be used. For example, a method of measuring the presence or an amount of LTBP-4 or DANCE using an antibody specific for LTBP-4 or DANCE by an immunological measuring method such as ELISA; and a method of quantitating an amount of a specified mRNA in a cell using a method such as a real time PCR method and a competitive PCR method are representatively exemplified. As the antibody used in the method, any of a monoclonal antibody and a polyclonal antibody may be used, and a commercially available product can be used.

A representative analysis method will be described below by referring to LTBP-4 as an example, but the method is not limited thereto.

<Real Time PCR Method>

Fibroblast is seeded on a 6-well plate, and cultured to the subconfluent state and, thereafter, a medium with a test substance dissolved therein is added to each well. After addition, culturing is performed for 48 hours to dissolve fibroblast, and extraction and purification of an intracellular total RNA is performed. Thereafter, a DNA complementary to a mRNA (cDNA) is synthesized using reverse transcriptase, an objective region is amplified by PCR using this cDNA as a template, and a process of production of the amplified product is monitored at a real time using a reagent for real time monitoring, and is analyzed. Examples of a primer specific for LTBP-4 used in the method include 28 kinds of primers supplied from Applied Biosystems. Specifically, when a human lung cell is used, the following two kinds of primer A or primer B can be used.

Primer A:
Forward: 5′-TGGCTGAGCCCTACGAGG-3
Reverse: 5′-CAGACTAAGCGGGCTGCAG-3′
Forward: 5′-GTGGAGCTGCCCTGTGTG-3′
Reverse: 5′-CCTGGTCTCGGAAGAGCTG-3′
Primer B:
Forward: 5′-CCCGCTCCGTTTATACAATG-3
Reverse: 55-AGGAAACCGTCCGGAC-3′

<Method of Using QuantiGene (Manufactured by PANOMICS)>

Fibroblast is seeded on a culturing plate etc., and cultured to the subconfluent state and, thereafter, a medium with a test substance dissolved therein is added to each well. Thereafter, a lysate of the cultured cell is recovered, and is added to a well having, as a stationary phase, a capture probe having a sequence complementary to a mRNA of LTBP-4. After the reaction, nonspecific binding is washed, a probe further having a complementary sequence to a mRNA of an objective gene bound to a stationary phase probe, and a labeling probe thereto are reacted, and a color developing reaction is performed using a substrate. An amount of gene expression is assessed by a color development intensity or the presence or the absence of color development.

<Method of Using Quantigene Plex>

This method is a method of using microbeads labeled with a fluorescent dye as a carrier to immobilize a capture probe having a sequence complementary to a mRNA onto these microbeads, and reading an incorporation ratio of a fluorescent dye, thereby, specifying a probe.

Specifically, fibroblast is seeded on a culturing plate etc., and cultured to the subcontinent sate and, thereafter, a medium with a test substance dissolved therein is added to each well. Thereafter, a lysate of the cultured cell is recovered, and is reacted with fluorescent microbeads onto which a capture probe having a sequence complementary to a mRNA of LTBP-4 is immobilized. After the reaction, non-specific binding is washed, a probe further having a complementary sequence to a mRNA of an objective gene bound to a capture probe, and a labeling probe thereto are reacted, and a color developing reaction is performed using a fluorescent substrate. Thereafter, a fluorescent intensity of the labeling probe bound to fluorescent microbeads, or a fluorescent intensity of fluorescent microbeads bound to a labeling probe is measured with Lurainex100.

<ELISA Method>

As the ELISA method, any of a direct adsorption method and a sandwich method can be applied. The direct adsorption method is useful in the case where an antibody to LTBP-4 is only one of a monoclonal antibody or a polyclonal antibody, while when there are a plurality of antibodies to LTBP-4, a more specific sandwich method is useful.

Among them, the direct adsorption method (direct ELISA method) is performed, for example, as follows. First, the supernatant (or cell lysate) of fibroblast cultured in a medium with a test substance dissolved therein is diluted with PBS etc., and this suspension is added to a well to adsorb onto a stationary phase at 4° C. overnight. Thereafter, the well is washed using PBS etc., and an anti-LTBP-4 antibody solution is added to each well to react at room temperature for 1 hour. Thereafter, the well is washed using PBS etc., a secondary antibody solution is added to react at room temperature for 1 hour, and a fluorescent intensity or a color development intensity after the reaction is measured.

In addition, the sandwich ELISA method is performed, for example, as follows. First, an anti-LTBP-4 monoclonal antibody solution is added to each well, and is adsorbed onto a stationary phase at 4° C. overnight. Thereafter, the well is washed using PBS etc., the supernatant (or cell lysate) of fibroblast cultured in a medium with a test substance dissolved therein is diluted with PBS etc., and this suspension is added to each well to react at room temperature for 1 hour. Thereafter, the well is washed using PBS etc., and an anti-LTBP-4 polyclonal antibody is added to each well to react at room temperature for 1 hour. Thereafter, the well is washed using PBS etc., a secondary antibody solution is added to react at room temperature for 1 hour, and a fluorescent intensity or a color development intensity after the reaction is measured.

<Method of Using Luminex100>

This method is a method of using, as a carrier, microbeads labeled with two kinds of fluorescent dyes, immobilizing a probe onto these microbeads, and reading an incorporation ratio of the fluorescent dyes, thereby, specifying the probe. A principle of the method is the same as that of the sandwich ELISA method.

Specifically, fluorescent beads to which a mono- or polyclonal antibody specific for LTBP-4 is bound (beads 1) are reacted with the supernatant (or cell lysate) of fibroblast cultured in a medium with a test substance dissolved therein. After the reaction, the reaction is washed using BPS etc., and further reacted with fluorescent beads to which a mono- or polyclonal antibody specific for LTBP-4 is bound (beads 2), which are different from the above beads. After the reaction, the reaction is washed, and a fluorescent intensity of beads 2 bound to beads 1, or a fluorescent intensity of beads 1 bound to beads 2 is measured with Luminex100.

<Direct Measurement>

After fibroblast is seeded on a cell culture or a culturing plate etc., a test substance is added, followed by culturing. Thereafter, cells are immobilized and blocking is performed using a blocking agent such as BSA. Thereafter, after this is washed using BPS etc., a mono- or polyclonal antibody specific for LTBP-4 is reacted for a constant time, and this is washed using PBS etc. Then, after a reaction with a secondary antibody, a fluorescent intensity or a color development intensity is measured using a device such as a microscope or IN cell Analyzer (cell function imager).

### EXAMPLES

Hereinafter, the present invention will be described more specifically by way of Examples, but the present invention does not undergo restriction by the following Examples, the present invention can be also implemented by appropriate alteration in such a range that can be in conformity with the gist described above and later, and all of them are included in the technical scope of the present invention.

### 1. Making of Recombinant LTBP-4, and Recombinant DANCE

An entire coding region of human LTBP-4 cDNA (GenBank accession number: AF051344) was cloned by a PCR method, and a FLAG tag and a 6×His tag were attached to a carboxyl terminal, and the resultant was subcloned into a pEF6 vector (purchased from Invitrogen). This expression vector was transfected into 293T cells, and a human LTBP-4 stably expressing cell strain was made using a Blasticidin-resistant gene as a marker. Recombinant LTBP-4 was purified from the serum free culture supernatant of the LTBP-4 stably expressing cell strain using Ni-NTA agarose (Qiagen), developed with SDS-PAGE, stained with Coomasie-Blue (CBB), and desalted by dialysis.

According to the same manner, recombinant DANCE was purified, developed with SDS-PAGE, stained with CBB, and desalted by dialysis. Herein, an entire coding region of human DANCE cDNA (GenBank accession number: AF112152) was cloned by a PCR method.

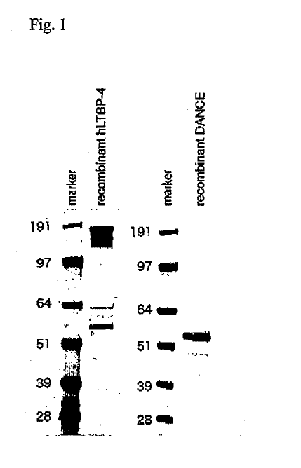

FIG. 1 shows a photograph of purified recombinant LTBP-4 (about 180 kD) and recombinant DANCE (about 58 kD) CBB-stained.

### 2. In Vitro Experiment in the Presence of Serum

(1) First, the following experiment was performed in order to verify that LTBP-4 is essential for formation of the elastic fiber by human skin fibroblast in the presence of serum.

(Experimental Material)

As a culture for subculturing, DMEM (Dulbecco's Modified Eagle Medium, purchased from Invitrogen), 10% bovine fetal serum (FBS, purchased from JRH Bioscience), 2 mM glutamine, 100 units/ml, penicillin, and 100 g/mL streptomycin were used. In addition, upon formation of the elastic fiber, DMEM/F12 (Dulbecco's Modified Eagle's Medium/Ham's Nutrient Mixture F12, purchased from Invitrogen), 10% FBS, 2 mM glutamine, 100 units/mL penicillin, and 100 g/mL streptomycin were used as an elastic fiber formation medium.

As human skin fibroblast, human skin fibroblast provided from Kyoto University Hospital Plastic Surgery was used as in the aforementioned patent publication 2 (WO 2006/082763 pamphlet).

For fluorescent immunostaining of the elastic fiber, as a primary antibody, an anti-elastin polyclonal antibody (purchased from Elastin Products Company) and an anti-DANCE monoclonal antibody 10A (made by immunizing a DANCE gene-deficient mouse with recombinant DANCE) were used and, as a secondary antibody, an Alexa488-anti-rabbit IgG antibody (purchased from Invitrogen), and an Alexa546-anti-mouse IgG antibody (purchased from Invitrogen) were used.

(Experimental Method)

Microscope Cover Glass (purchased from Fisherbrand) was placed on a bottom of a 24-well plate, human fibroblast was seeded thereon at 7.5×10⁴ per well, and cultured at 37° C. and 5% CO₂ on the medium for subculturing. After washed with PBS on 3 day of culturing, the medium was exchanged with the elastic fiber formation medium and, subsequently, cells were cultured at 37° C. and 5% CO₂ for 2 weeks.

After culturing for 2 weeks, fluorescent immunostaining of the elastic fiber was performed. More particularly, after washing three times using 1 mL PBS, cells were fixed at −20° C. for 30 minutes using 100% methanol. After further washing using PBS, this was blocked at room temperature for 30 minutes using PBS containing 2% BSA, and incubated with the primary antibody at room temperature for 1 hour or more. Then, this was washed with PBS, and incubated with the secondary antibody at room temperature for 1 hour. After washing with PBS, this was fixed at room temperature for 10 minutes using 4% paraformaldehyde, and washed again with PBS and, thereafter, a sample was mounted on a slide glass with Vectashield (purchased from Vector Laboratories) containing DAPI. Observation was performed using a fluorescent microscope, DMIRE2 (Leica).

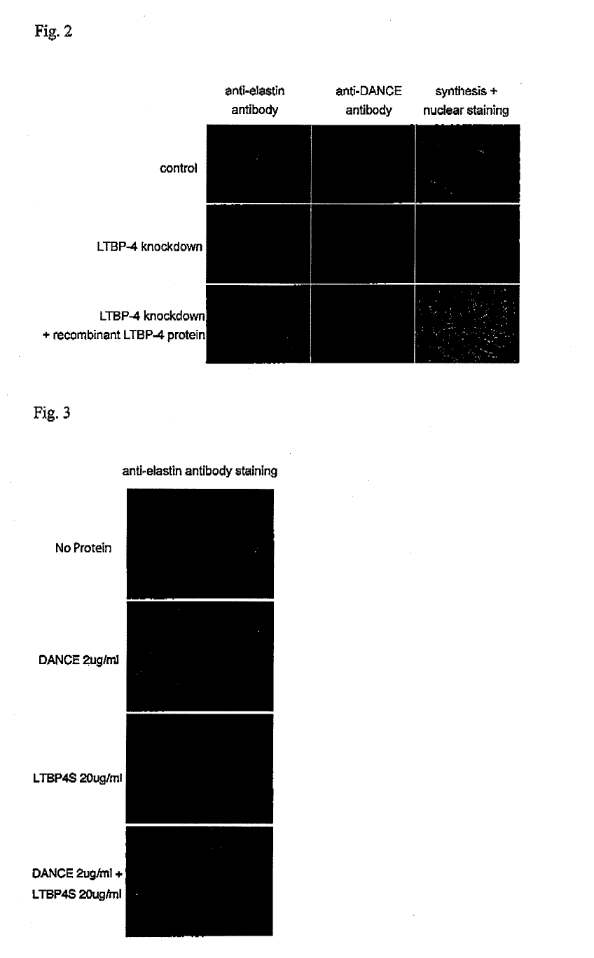

These fluorescent immunostaining photographs are shown in FIG. 2 (see uppermost column, control). A rightmost column of FIG. 2, for reference, shows synthetic results of staining of an anti-elastin polyclonal antibody and an anti-DANCE monoclonal antibody 10A. From FIG. 2, formation of the elastic fiber was confirmed when any antibody was used. That is, it was seen that when human skin fibroblast is cultured in the presence of serum, an elastic fiber which are stained with both of an anti-elastin antibody and an anti-DANCE antibody is formed.

Then, using cells in which LTBP-4 was knocked down by gene knockdown using RNA interference, fluorescent immunostaining was performed according to the aforementioned manner to investigate formation of an elastic fiber. Gene knockdown was performed by introducing Stealth Select siRNA (purchased from Invitrogen) to a LTBP-4 gene into a human skin fibroblast culture, according to the protocol of RNAiMAX (purchased from Invitrogen). Then, culturing was performed for 2 weeks as described above, and fluorescent immunostaining of the elastic fiber was performed.

These fluorescent immunostaining photographs are shown in FIG. 2 (see LTBP-4 knockdown). From FIG. 2, formation of the elastic fiber was confirmed little when any antibody was used, and fibrous sedimentation of not only elastin, but also DANCE disappeared. That is, it was seen that when LTBP-4 is knocked down with a siRNA, formation of the elastic fiber becomes not to be seen.

From the above experimental results, it was confirmed that LTBP-4 is essential for formation of the elastic fiber by human fibroblast in the presence of serum.

(2) Then, it was investigated that the recombinant LTBP-4 has the ability to regenerate the elastic fiber by human skin fibroblast in the presence of serum.

After LTBP-4 was knocked down with a siRNA by the method of (1), recombinant LTBP-4 was added to an elastic fiber formation medium at a concentration of 10 μg/mL, followed by culturing for 2 weeks. After culturing, fluorescent immunostaining of the elastic fiber was performed as in (1).

These fluorescent immunostaining photographs are shown in FIG. 2 (see LTBP-4 knockdown+recombinant LTBP-4). From FIG. 2, when recombinant LTBP-4 was added after MP-4 had been knocked down with a siRNA, an elastic fiber was formed more as compared with the case of knockdown, when any antibody was used. That is, it was seen that the elastic fiber regenerating ability which has been inhibited by LTBP-4 knockdown is recovered by addition of recombinant LTBP-4.

Further very interestingly, when recombinant LTBP-4 was added, far more elastic fiber was formed even as compared with a control of no knockdown. Thereupon, it was also confirmed that fibrous sedimentation is restored also in cell-derived DANCE as in elastin. Since it was found out that the elastic fiber regenerating activity of LTBP-4 is extremely strong, far exceeding the activity by serum itself, like this, it was strongly suggested that the LTBP-4-containing regenerating agent of the present invention is extremely useful for regeneration of the elastic fiber.

### 3. Experiment Under Serum Free

The elastic fiber forming abilities when human skin fibroblast was cultured in a serum free medium using recombinant LTBP-4 alone, recombinant DANCE alone, and both of them were compared and studied below.

Herein, the elastic fiber formation medium containing no serum, that is, DMEM/F12, 2 mM glutamine, 100 units/mL penicillin, and 100 g/mL streptomycin were used, and human fibroblast was seeded at 7.5×10⁴ per well, and cultured at 37° C. and 5% CO₂ for 2 weeks on the serum free medium. Thereafter, fluorescent immunostaining was performed using an anti-elastin antibody as in (1). As a result, little formation of the elastic fiber was seen under a serum free medium (see “No protein” of FIG. 3).

Then, recombinant DANCE was added to the serum free medium at a concentration of 2 μg/mL, and human fibroblast was cultured as described above, followed by the fluorescent immunostaining. As a result, by addition of recombinant DANCE, slight formation of the elastic fiber was observed (see “DANCE 2 μg/mL” of FIG. 3).

Similarly, recombinant LTBP-4 was added to the serum free medium at a concentration of 20 μg/mL, and human fibroblast was cultured as described above, followed by fluorescent immunostaining. As a result, by addition of recombinant LTBP-4, slight formation of the elastic fiber was also observed (see “LTBP-4S 20 μg/mL” of FIG. 3).

Then, 2 μg/mL recombinant DANCE and 20 μg/mL recombinant LTBP-4 were added to the serum free medium at the same time, and human fibroblast was cultured as described above, followed by fluorescent immunostaining. As a result, when these were added at the same time, far more elastic fiber was formed as compared with the case where each was added alone (see “DANCE 2 μg/mL+LTBP-4S 20 μg/mL” of FIG. 3).

From the foregoing experimental results, it was confirmed that, under the serum free medium, DANCE and LTBP-4 work cooperatively to promote formation of the elastic fiber.

### 4. Binding of DANCE and LTBP-4

Herein, in order to investigate interaction of DANCE and LTBP-4, the following coimmunoprecipitation experiment was performed.

First, DANCE in which a FLAG tag had been attached to a carboxyl terminal of full length DANCE, and LTBP-4 in which a Myc tag had been attached to a carboxyl terminal of full length LTBP-4 were incorporated into a pEF6 vector, respectively. After these were transfected into 293T cells separately using Lipofectamine Plus (purchased from Invitrogen), cells were cultured on a serum free medium for 2 days to secrete DANCE-FLAG and LTBP-4-Myc in the supernatant. The thus obtained culture supernatant was mixed, and allowed to stand on an ice for 1 hour, and immunoprecipitation (IP) was performed with anti-FLAG agarose (purchased from Sigma). The immunoprecipitate was developed with SDS-PAGE, and detected by Western blotting using an anti-Myc antibody and an anti-FLAG antibody.

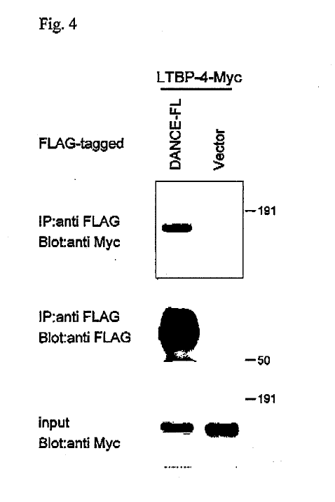

The results thereof are shown in FIG. 4. In FIG. 4, an upper figure shows the result of detection with an anti-Myc antibody; a middle figure shows the results of detection with an anti-FALL antibody; a lower figure shows the result of detection of a solution before immunoprecipitation with an anti-Myc antibody. From FIG. 4, LTBP-4-Myc bound to DANCE-FLAG which had been immunoprecipitated with anti-FLAG agarose. In addition, since LTBP-4-Myc is not detected on a vector lane, it is seen that binding of LTBP-4-Myc to an anti-FLAG agarose is specific binding via DANCE-FLAG. From the experimental results, it was strongly suggested that LTBP-4 directly binds to DANCE to exert the elastic fiber regenerating ability.

### 5. In vivo Experiment of LTBP-4 Plus DANCE

The following experiment was performed to demonstrate that the combined use of recombinant LTBP-4 and recombinant DANCE makes it possible to regenerate elastic fibers even in vivo.

(Experimental Method)

Four Japanese house rabbits (9 weeks old, female) purchased from Oriental Yeast Co., Ltd. were habituated for a constant period, after which the back of each house rabbit was sheared with an electric shaver, and the house rabbits thus treated were used in the experiment.

The above four house rabbits were classified into the following two groups (i.e., n=2): test substance-treated group and the control substance-treated group, and test substance or control substance were intradermally administered to the sheared portion (about 2 cm×2 cm) of each house rabbit at an interval of three times per week. More specifically, physiological saline solution was administered as a control substance to the control substance-treated group at an amount of 200 μL per portion. On the other hand, a preparation obtained by mixing recombinant LTBP-4 and recombinant DANCE at a ratio of 1:1 to contain each recombinant protein at an amount of 1 μg in 200 μL was administered as a test substance to the test substance-treated group at an amount of 200 μL per portion.

On the fourth day from the completion of administration for six weeks, the house rabbits were sacrificed, and two skins were collected from each administered portion of the test substance-treated group and the control substance-treated group. The skins on the one hand were each embedded in OCT compound (Optimal Cutting Temperature Compound) while remaining unfixed, and then stored at −80° C. Then, a frozen section of 8 μm in thickness was prepared for each skin, followed by EVG (Elastica Van Gieson) staining, and examined for the presence or absence of elastic fibers.

Further, the skins on the other hand were each cut into a 5 mm square piece, and each skin piece was transferred into a glass vial, to which 1.5 mL of 6N HCL was added, and each skin piece was heat treated in vacuum at 110° C. for 48 hours. After the lapse of 48 hours, each skin piece was sprayed with N₂ gas, while warming to a temperature of 80° C., to remove the solvent (HCL), and then used as a sample for measurement of desmosine and isodesmosine, which are amino acids inherent to elastin. Desmosine and isodesmosine are amino acids formed from lysine residues in tropoelastin, which is an elastin precursor, at the process of elastin biosynthesis, and they have been used as indicator amino acids because of absence in other proteins such as collagen.

The measurement of desmosine and isodesmosine was requested to Shimadzu Techno-Research Inc., and performed by the use of an LC-MS/MS apparatus (HPLC: LC20A system available from Shimadzu Corporation, MS/MS: API-3200 available from AB SCIEX). More specifically, the above each sample was dissolved in 100 μl of 0.05% heptafluorobutyric acid solution directly added thereto, and the solution was separated by centrifugation at 1,200 rpm for 5 minutes. The supernatant thus obtained was injected into the above LC-MS/MS apparatus, and measured for desmosine and isodesmosine. At that time, desmosine and isodesmosine purchased from Elastin Products Co., Inc. were used as blanks.

On the other hand, for comparison, L-methionine, L-valine, and L-tyrosine were also measured by the above method. At that time, L-methionine, L-valine, and L-tyrosine purchased from Wako Pure Chemical Industries, Ltd. were used as blanks. In the present experiment, the measurement of these amino acids, in particular, was intended for comparison with the amounts of desmosine and isodesmosine. This is because the comparison of relative amounts to these amino acids makes it possible to compare the abilities of forming elastic fibers in both groups with higher accuracy than the comparison of absolute amounts of desmosine and isodesmosine.

In the present experiment, the amounts (ng) of desmosine and isodesmosine relative to each 1 μg of the above three amino acids (i.e., L-methionine, L-valine, and L-tyrosine) were calculated, and the above amounts for the test substance-treated group and the control substance-treated group were compared with each other.

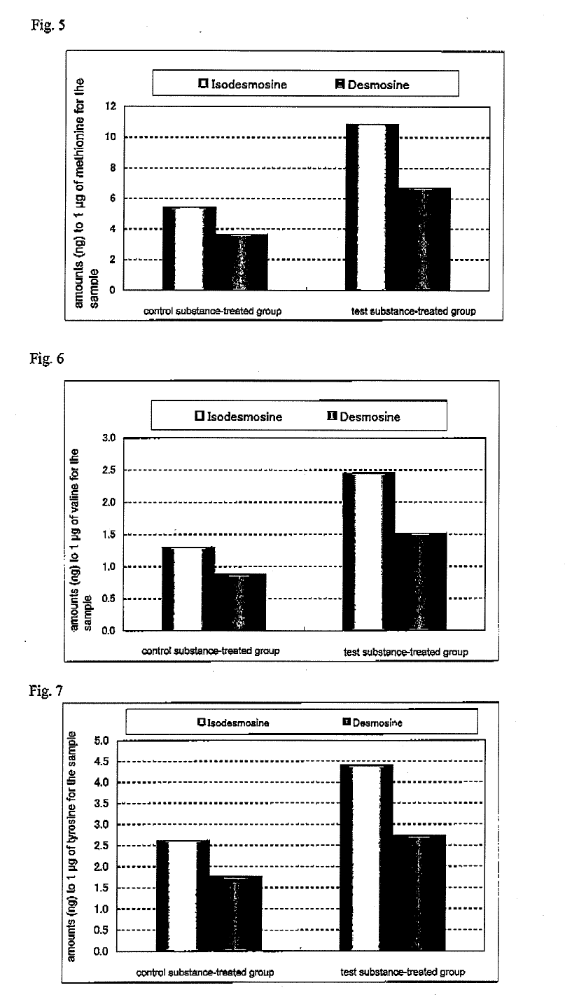

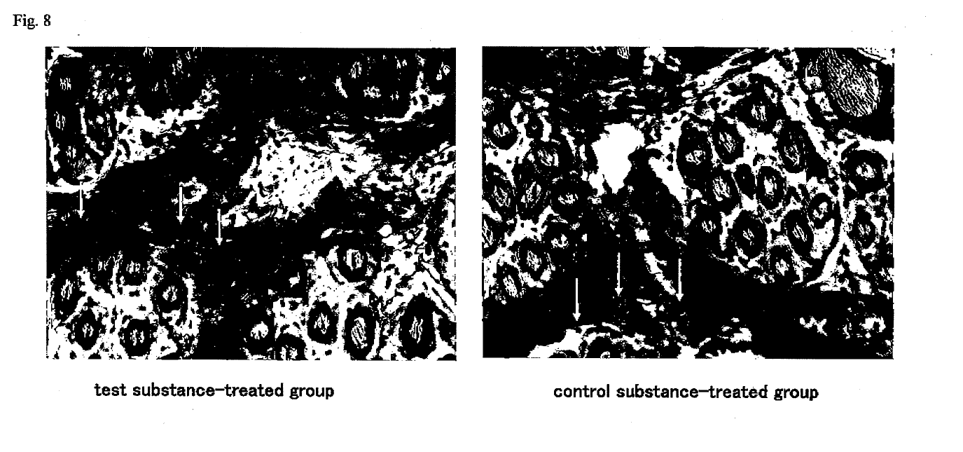

These results are shown in FIGS. 5 to 7. In each of FIGS. 5 to 7, the results of isodesmosine are shown on the left side for each treated group, and the results of desmosine are shown on the right side for each treated group. In addition, FIG. 8 shows the result of EVG staining. In FIG. 8, elastic fibers are indicated by arrows.

First, reference is made to FIG. 8. From FIG. 8, in the EVG staining of house rabbit frozen sections, it is found that thicker and steadier elastin fibers were formed in the test substance-treated group as compared with in the control substance-treated group.

Then, references are made to FIGS. 5 to 7. The amounts of desmosine and isodesmosine relative to each amino acid, i.e., L-methionine, L-valine, or L-tyrosine, were increased, despite the kind of amino acid, in the test substance-treated group as compared with in the control substance-treated group. More specifically, the amount of desmosine was approximately about 1.5 to 1.8 times increased, while the amount of isodesmosine was approximately about 1.7 to 2.0 times increased. In other words, it was confirmed that the ratios of desmosine and isodesmosine inherent to elastic fibers are increased by the combined use of recombinant LTBP-4 and recombinant DANCE.

From the above results of FIGS. 5 to 8, it was found that the combined used of recombinant LTBP-4 and recombinant DANCE makes it possible to promote the formation of elastic fibers even in vivo.

(Discussion of Molecular Mechanism)

A series of experimental results described above (particularly, experimental result in which fibrous sedimentation of not only elastin but also DANCE disappeared by knockdown of LTBP-4, while when recombinant LTBP-4 was added thereto, fibrous sedimentation of not only elastin but also DANCES was increased, and the experimental result in vitro by using LTBP-4 plus DANCE) strongly suggests that LTBP-4 is essential, in order that DANCE is sedimented on a microfibril constituting the elastic fiber. That is, as a molecular mechanism of elastic fiber regeneration by LTBP-4, first, a model in which LTBP-4 is sedimented on a microfibril, DNACE is induced by a mark of this LTBP-4, DANCE binds on LTBP-4, and elastin further binds on DANCE, is thought. The aforementioned binding of DANCE and elastin, and direct binding of DANCE and LTBP-4 shown herein strongly support this model.

According to this model, formation of the elastic fiber is restricted in a region where any one of LTBP-4 and DANCE is deficient. Actually, in serum free culturing of human skin fibroblast, the elastic fiber regenerating activity was seen slight upon addition of recombinant LTBP-4 alone, or addition of recombinant DANCE alone, while when both of them were added, more elastic fiber was formed synergistically. The ability to promote the formation of elastic fiber by the combined used of recombinant LTBP-4 and recombinant DANCE was seen in vivo similarly. In application to elastic fiber regeneration, since both of LTBP-4 and DANCE can be deficient depending on an administration site, it is suggested that more therapeutic effect can be expected in a cocktail of recombinant LTBP-4 and recombinant DANCE, more than recombinant LTBP-4 alone.

Sequence listing (please see the attached text file)

1. A method of regenerating an elastic fiber, comprising bringing an elastic fiber regenerating agent containing LTBP-4 into contact with a cell having the fiber regenerating ability.

2. The method according to claim 1, wherein the agent further contains DANCE.

3. The method according to claim 1, wherein the agent further contains a LTBP-4 expression potentiating factor and/or a DANCE expression potentiating factor.

4. The method according to claim 2, wherein the agent further contains a LTBP-4 expression potentiating factor and/or a DANCE expression potentiating factor.

5. The method according to claim 1, wherein the agent is contacted with the cell in the presence of serum.

6. The method according to claim 2, wherein the agent is contacted with the cell in the presence of serum.

7. The method according to claim 3, wherein the agent is contacted with the cell in the presence of serum.

8. The method according to claim 4, wherein the agent is contacted with the cell in the presence of serum.

9. A method of screening a LTBP-4 expression potentiating factor, comprising using an expression amount of LTBP-4 or a mRNA encoding LTBP-4 as an index.

10. A method of screening an elastic fiber regenerating ability, comprising using an expression amount of LTBP-4 or a mRNA encoding LTBP-4 as an index.

11. The method of screening an elastic fiber regenerating ability according to claim 10, wherein an expression amount of DANCE or a mRNA encoding DANCE is further used as an index.
